(19) United States
(12) Patent Application Publication
(10) Pub. No.: US 2007/0224164 A1
(43) Pub. Date: Sep. 27, 2007

(54) Degradable nanogel for drug delivery

(76) Inventors: Maciej Radosz, Laramie, WY (US); Youqing Shen, Laramie, WY (US); Edward Alva VanKirk, Laramie, WY (US); William James Murdoch, Laramie, WY (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/599,531

(22) Filed: Nov. 14, 2006

Related U.S. Application Data
(60) Provisional application No. 60/736,540, filed on Nov. 14, 2005.

(51) Int. Cl. A61K 31/765; C08F 20/00

(52) U.S. Cl. 424/78.37; 525/438

(57) ABSTRACT

A nano-sized hydrogel is made of a water-soluble chain containing carboxylic acid moieties and polyethylene side chains. Such a nanogel is applicable as a cancer-drug delivery agent or an imagining agent, where either a cancer drug, such as cisplatin, or an imaging agent, such as Gd3+. The complexation of the cancer drug or the imaging agent with the carboxyl moieties leads to the hydrogel formation.

This application claims priority to U.S. Patent Application Ser. No. 60/736,540, filed Nov. 14, 2005.

### BACKGROUND OF THE INVENTION

The present invention relates generally to methods of treating cancer and, more specifically, to a biodegradable core-shell nano-sized gels (nanogels) that can be used for cancer-drug delivery or cancer-tissue imaging.

Cancer is the second leading cause of death in the United States. Each year more than 1.2 million Americans are diagnosed with cancer, and less than half can survive five years. Annual medical costs for cancer treatment account for billions of dollars in the US alone. Chemotherapy, which uses chemical agents (anticancer drugs) to kill cancer cells, is one of the primary methods of cancer treatment. Unfortunately, these anticancer drugs have limited selectivity for cancer and are inherently toxic to both cancer and normal tissues. As a result, anticancer drugs can cause severe side effects and damage to healthy tissues. For example, cisplatin is a well-known metal complex that exhibits high antitumor activity [Rosenberg et al., 1969; Takahara et al., 1995]. However, it has significant toxicity, in particular, acute as well as chronic nephrotoxicity [von Hoff et al., 1979; Pinzani et al., 1994]. Other common side effects of anticancer drugs include decrease in the number of white blood cells (increasing risk of infection), red blood cells (losing energy) and platelets (risk for bruising and bleeding) as well as nausea, vomiting, hair loss, etc. Furthermore, the high glomerular clearance of the anticancer drugs leads to an extremely short circulation period in the blood compartment [Siddik et al., 1987].

Most importantly, treatments in conventional dosage form of these drugs may lead to initial cancer regression, but soon the cancer becomes insensitive to the drugs, causing cancer progression and death. The primary reason for the treatment failure is cancer's intrinsic and acquired drug resistance [Pastan and Gottesman, 1991; Gottesman, 2002]. When a conventional drug dose is administered intravenously, the drug molecules distribute throughout the body and some drug molecules reach the cancer interstititium. Some are taken up by cancer cells via diffusion, transport and endocytosis. On the other hand, cancer cells have various mechanisms by which they become resistant to the drugs, such as loss of a cell surface receptor or transporter for a drug to slow down the drug influx, specific metabolism of a drug, alteration by mutation or drug detoxification to consume the drugs, and the like [Gottesman, 2002]. A major mechanism of multidrug resistance is an energy-dependent drug efflux transporter, the P-glycoprotein (P-gp) pump located in cell membrane [Gottesman, 2002]. P-gp pumps are very efficient in detecting and binding a large variety of hydrophobic drugs as they enter the plasma membrane. These pumps then transport the drugs out of the cells [Bogman et al., 2001; Gottesman, 2002]. As a consequence of the slowed drug entry but efficient drug removal by the P-gp pumps and the drug consumption by other forms of drug resistance, the effective drug concentration in cytoplasm is well below the cell-killing threshold, resulting in a limited therapeutic efficacy.

The goal of this invention is to increase the drug selectivity for cancer and to overcome the cancer drug resistance toward enhanced therapeutic efficacy and reduced toxicity to healthy tissue.

### SUMMARY OF THE INVENTION

The present invention relates to degradable nanogels for the delivery of drugs. The nanogels are made of a polymer that has a water-soluble polymer chain containing carboxylic acid moieties and poly(ethylene glycol) (PEG) or other water-soluble polymer chains attached to the polymer chain and having the generic structure of FIG. 1. The water-soluble side chains have a molecular weight preferably between 300 and 50,000 and most preferably between 2000 and 5000. The polymers are useful as drug carriers and as imaging agents, and can overcome cancer drug resistance leading to enhanced therapeutic efficacy and reduced toxicity of the cancer drug to healthy tissue. In a particular application of the present invention, the polymer complexes with the cancer drug cisplatin to form core shell type gels with a size from 1 nm to 2000 nm.

### BRIEF DESCRIPTION OF THE FIGURES

FIG. 1 is a scheme of the generic structure of the polymeric compound claimed in this invention.

FIG. 2 is a scheme of the synthesis of 2,2-bis(acryloxymethyl)propionic acid.

FIG. 3 is a scheme of the synthesis of PEG macromonomer and poly (β-aminoester)-graft-PEG by polycondensation of 2,2-bis(acryloxymethyl)propionic acid.

FIG. 4 is a scheme of the formation of core-shell nanogel of the poly (β-aminoester) and CDDP.

FIG. 5 is a graph of the size distribution of the CDDP-induced nanogels.

FIG. 6 is a graph of the cytotoxicity of the polymers (P2K-25P, P25K-50P) and their nanogels at COOH/Pt of 3 (P2K-25-3 and P2K-50-3); cisplatin equivalent dose is 0.25.

FIG. 7 is a graph of tumor formation in SKOV-3 xenografted nude mice treated with cisplatin and with the nanogels (P2K-25-3 and P2K-50-3); A is the average number of tumors present per cm of mesentery tissue; B is the average diameters of tumors present; C is the tumor area as a ratio of the total area.

### DETAILED DESCRIPTION OF PREFERRED EMBODIMENTS

Drug Example

Cis-dichiorodiamineplatinum (II) (cisplatin or CDDP) is an example of an anticancer drug that can induce cancer drug resistance. [Scanlon, 1991.] Kataoka and co-workers incorporated cisplatin into block copolymer micelles to circumvent the drug resistance. The micelle-encapsulated cisplatin had an improved cytotoxicity. [Noshiyama, 2003; Nishiyama, 1999; Nishiyama, 2001.]

Nanogel Example

We synthesized a water-soluble carboxylic acid-containing poly(ester) grafted with PEG side chains. In water, CDDP complexes with the carboxylic acid of the poly(ester) and forms nanogel domains of about 100-200 nm that can be used for controlled delivery of CCDP or other drugs to cancer tissue.

Synthesis of 2,2-bis(acryloxymethyl)propionic Acid:

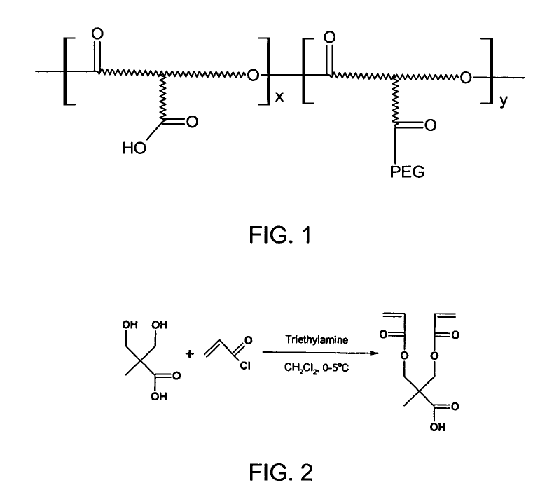

The reaction scheme for producing 2,2-bis(acryloxymethyl)propionic acid is illustrated in FIG. 1. 2,2-bis(hydroxymethyl) propionic acid (10.1 g, 0.075 mol) was stirred at 0-5° C. in dried dichloromethane. Triethylamine (15.2 g, 0.15 mol) was added with stirring. Acryloyl chloride (13.64 g, 0.15 mol) was added dropwise to the solution in 1.5 h. The resulting mixture was stirred for 0.5 h and then filtered. The filtrate was concentrated by removal of the solvent under vacuum and the residue was dissolved in 50 ml Na₂CO₃ aqueous solution (10% w/v). Hydrochloric acid (6N) was then dropped in with vigorous stirring, until the pH reached 2.0. Finally, dichloromethane (3×50 ml) was added to the solution. The dichloromethane solution was then concentrated by removal of the solvent under vacuum. The crude product was recrystallized with ethyl acetate/hexane mixed solvent and yielded the 2,2-bis(acryloxymethyl)propionic acid as white crystals. ¹H NMR (CDCl₃, 400 MHz) δ=11.47 (s, 1H); 5.7-6.8 (m, 6H), 4.4 (s, 4H), 1.38 (s, 3H).

Synthesis of PEG Macromonomer:

The reaction scheme for synthesizing the PEG macromonomer is illustrated in FIG. 2. Poly(ethylene glycol) methy ether (Mn ca. 2000) (10.0 g, 0.005 mol) and 2,2-bis(acryloxymethyl)propionic acid (3.03 g, 0.0125 mmol), N,N-dicyclohexylcarbodiimide (DCC) (2.588 g, 0.0125 mol), 4-dimethylaminopyridine (0.152 g, 0.00125 mol) and a polymerization inhibitor were dissolved in 50 ml of dry dichioromethane and stirred at room temperature for 72 h. The mixture was then filtered and washed with a small volume of dichioromethane. The filtrate was precipitated in ether, and purified by reprecipitation to give the product as white powder.

Synthesis of Poly(ester)-graft-PEG:

The reaction scheme for synthesizing poly(ester)-graft-PEG is illustrated in FIG. 2. 2,2-Bis(acryloxymethyl)propionic acid (0.242 g. 0.001 mol), PEG macromonomer (2.224 g, 0.001 mol) and piperazine (0.172 g. 0.002 mol) were dissolved in 20 ml of N,N-dimethylformamide and stirred at room temperature for 7 days. The molecular weight and polydispersity of the polymer were measured by GPC and calibrated with PEG standards.

Preparation of Nanogels:

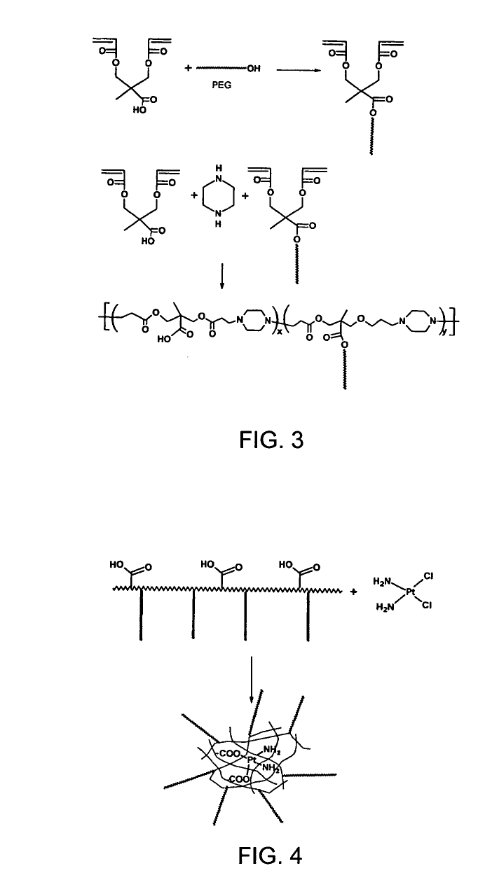

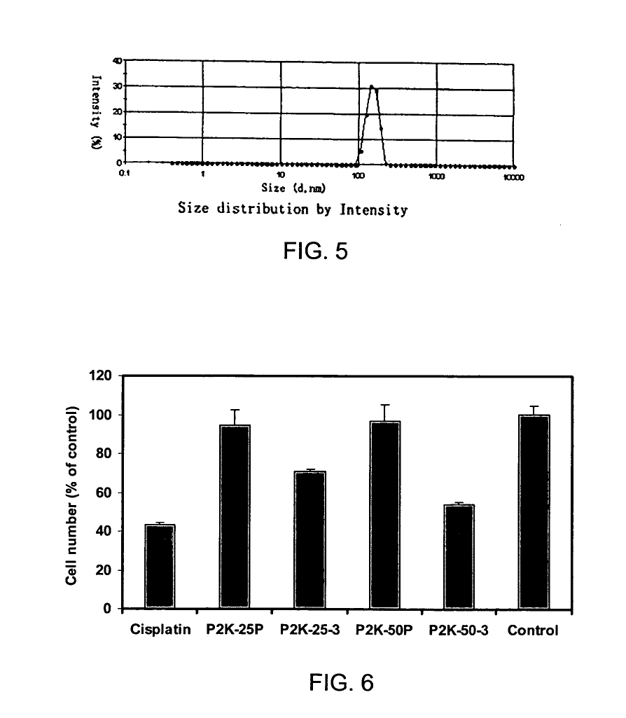

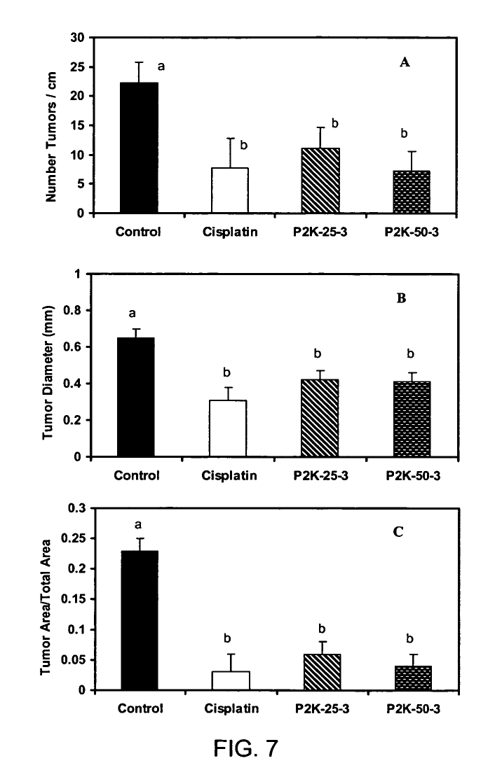

The reaction scheme for synthesizing the nanogels is illustrated in FIG. 3. Poly(β-aminoester)-graft-PEG and CDDP were dissolved in distilled water ([COOH]/[CDDP]=7.4) and stirred for a certain period of time at room temperature or heated at 70° C. for 10 minutes and then kept stirring at room temperature for 12 h. The size of the formed nanogels was evaluated by Nanosizer (Malvern Instruments).

Poly(ester)-graft-PEG Examples

The graft copolymers with PEG side chains (Mₙ=2000) was prepared by direct condensation. The PEG chain density was controlled by the molar ratio of 2,2-bis(acryloxymethyl)propionic acid to the PEG macromonomer. The pendent carboxyl acid groups are used for the complexation with CDDP. Composition of the copolymer was measured with ¹HNMR by the ratio of the OCH₂CH₂ signal intensity in PEG (3.60 ppm) and that of CH₃⁻ in 2,2-bis(acryloxymethyl)propionic acid (1.38 ppm). Polyester-graft-PEG with varied contents can be synthesized in a similar method.

TABLE 1
Poly(ester)-graft-PEG copolymers with different PEG
chain lengths and PEG unit feed ratios

| Composition | P2K-25 | | P2K-50 | | P5K-25 | | P5K-50 | |
|---|---|---|---|---|---|---|---|---|
| (mol %) | feed | NMR | feed | NMR | feed | NMR | feed | NMR |
| mPEG | 25 | 26.5 | 50 | 44.4 | 25 | 25.2 | 50 | 36.2 |
| Carboxyl acid | 75 | 73.5 | 50 | 55.6 | 75 | 74.8 | 50 | 63.8 |

Characterization of the CDDP-Induced Formation of Nanogels.

The PEG grafted polymer and CDDP reacted in distilled water. The carboxylic groups of the graft-copolymer complexed with CDDP, and thus the polymer was crosslinked to form the gel core, while the PEG chains formed the hydrophilic corona (FIG. 3). The average size was 220-240 nm (FIG. 4). Heating can decrease the size of the nanogel. When heated at 70° C. for ten minutes, the average size of the nanogels was reduced to 150-160 nm. The nanogels are negatively charged. The negatives and the PEG outer layer impart “stealth properties” to the nanogels suitable for in vivo drug delivery to cancerous tissues via the EPR effect. Particularly, the polymers alone showed no or little cytotoxicity.

The cisplatin-containing nanogels had low in vitro cytotoxicity to SKOV-3 ovarian cancer cells compared to free cisplatin. Similar phenomena were reported in the cisplatin-containing micelles and other water soluble cisplatin conjugates (Cabral et al. 2005; Nishiyama et al. 2003a; Nishiyama et al. 2003b; Nishiyama et al. 1999). However, the nanogels had similar in vivo anticancer activity to cisplatin tested in nude mice inoculated with SKOV-3 tumors. Grafting targeting groups to the nanogels is expected to further increase the anticancer activity.

In conclusion, the reaction of CDDP and PEG-grafted copolymer in distilled water led to the spontaneous formation of CDDP-incorporated micelles. The size of the micelles can be reduced by heating. These nanogels are useful for drug delivery.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

Bogman, K.; Peyer, A. K.; Torok, M.; Kusters, E.; Drewe, J. HMG-CoA reductase inhibitors and P-glycoprotein modulation, Br. J. Pharmacol. 132 (2001), 1183-92.
Gottesman, M. M. Mechanisms of cancer drug resistance, Annu. Rev. Med. 53 (2002), 615-627.
Nishiyama, N., Yokoyama, M., Aoyagi, T., Okano, T., Sakurai, Y., Kataoka, K., Langmuir, 15(1999), 377-383.
Nishiyama, N., Kato, Y., Sugiyama, Y., Kataoka, K., Pharm. Res., 7(2001), 1035-1041.
Nishiyama, N., Okazaki, S., Cabral, H., Miyamoto, M., Kato, Y., Sugiyama, Y., Nishio, K., Matsumura, Y., Kataoka, K., Cancer Res., 63(2003), 8977-8983.
Pastan, I. and Gottesman, M. M. Multidrug resistance, Annu. Rev. Med. 42 (1991), 277-286.
Pinzani, V.; Bressolle, F.; Hang, L. J.; Galtier, M.; Blayac, J. P.; Balmes, P. Cisplatin-induced renal toxicity and toxicity-modulating strategies—a review, Cancer Chemother. Pharmacol. 35(1994), 1-9.
Rosenberg, B.; VanCamp, L.; Trosko, J. E.; Mansour, V. H. Platinum compounds: a new class of potent antitumor agents, Nature 222(1969), 385.
Scanlon, K. J.; Kashani-Sabet, M.; Tone, T.; Funato, T. Pharmacol. Therap. 52(1991), 385-406.
Takahara, P. M.; Rosenzweig, A. C.; Frederick, C. A.; Lippard, S. J. Crystal-structure of double-stranded DNA containing the major adduct of the anticancer drug cisplatin, Nature 377(1995), 649-652.
Von Hoff, D. D.; Schilsky, R.; Reichert, C. M.; Reddick, R. L.; Rozencweig, M.; Young, R. C.; Muggia, F. M. Toxic effects of cis-dichlorodiammineplatinum(II) in man, Cancer Treat. Reports 63(1979), 1527-1531.

We claim:

1. A polymeric compound, comprising:
(a) a water-soluble polymer chain backbone containing carboxylic acid moieties;
(b) water-soluble polymer chains, optionally including poly(ethylene glycol), attached to the polymer chain; and
(c) having the generic structure

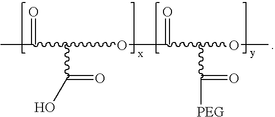

2. A polymer as defined in 1, wherein the polymer chain backbone comprises biodegradable polymers selected from the group consisting of polyesters and polycarbonates.

3. A polymer as defined in 1, wherein the side water-soluble chain comprises PEG or, optionally, a water-soluble polymer with a molecular weight from 300 to 50,000, and preferably from 2000 to 5000.

4. A polymer as defined in 1, wherein the carboxylic groups on the polymer side chains are randomly distributed along the polymer chain backbone.

5. A polymer as defined in claim 1, wherein the carboxylic groups on the polymer chain form sequences of varying degree of blockiness.

6. A method for transporting a drug in a living organism, comprising attaching the drug to a polymer as defined in 1.

7. A method as defined in claim 6, wherein the drug comprises cisplatin and wherein the cisplatin is complexed with the polymer to form nano-sized gels for cisplatin delivery.

8. A method for improving the efficacy of an image of a living organism, comprising administering an effective amount of a polymer as defined in claim 1.

9. A method as defined in 8, wherein an imaging agent is complexed with the polymer.

10. A method as defined in claim 9, wherein the imaging agent comprises Gd3+.

11. A polymer as defined in claim 1, wherein the water-soluble polymer chains attached to the polymer background comprise polyethylene glycol.
